(19) United States
(12) Patent Application Publication
(10) Pub. No.: US 2005/0055054 A1
(43) Pub. Date: Mar. 10, 2005

(54) Binding cloth for medium or low frequency wave massage

(76) Inventor: Tsung-I Yu, Taipei (TW)

Correspondence Address: BACON & THOMAS, PLLC, 625 SLATERS LANE, FOURTH FLOOR, ALEXANDRIA, VA, 22314, US

(21) Appl. No.: 10/658,386

(22) Filed: Sep. 10, 2003

(51) Int. Cl. A61N 1/00

(52) U.S. Cl. 607/2

(57) ABSTRACT

A binding cloth for medium or low frequency wave massage includes a plurality of conductive strips secured within a binding cloth on selected locations. Each conductive strip has a first end connected with a conductive plate and a second end connected with a conductive button. The conductive buttons are concentrated on a selected location of the binding cloth for being buckled up with a plurality of corresponding conductive buttons on an electro-stimulating controller so that the controller is mounted onto the binding cloth. Having the conductive plates applied to the human body, the electro-stimulating controller activates the binding cloth to provide the effect of medium or low frequency wave massage.

### BACKGROUND OF THE INVENTION

(a) Field of the Invention

The present invention relates to devices for medium of low-frequency wave massage, and more particularly to a binding cloth for medium or low frequency wave massage that is controlled by an electro-stimulating controller for achieving the effects of massage and fat reduction.

(b) Description of the Prior Art

The devices for medium or low frequency wave massage in the prior art are mainly composed of a controller and electrodes made of silica gel (or conductive gel or electrode cups made of silica gel). Those devices are disadvantageous in high production cost and low electrical conductivity and can only be applied in special occasions. Especially, silica gel must be smeared over body portions the electro-stimulating massage is applied to, which inevitably causes discomfort and cleansing problem to a user. Furthermore, silica-gel plates and silica-gel cups are both hard objects, which are heavy and uncomfortable to wear, substantially reducing the portability thereof.

### SUMMARY OF THE INVENTION

Accordingly, the primary objective of the present invention is to provide a binding cloth for medium or low frequency wave massage, which improves binding cloths such as waist belt, elbow guard and knee guard by incorporating a plurality of conductive strips therein. Each conductive strip has a first end connected with a conductive plate and a second end connected with a conductive button. The conductive plates are to be applied to the human body. The conductive buttons are concentrated on a selected location of the binding cloth for being buckled up with a plurality of corresponding conductive buttons on an electro-stimulating controller so that the controller is mounted onto the binding cloth. The electro-stimulating controller charges the conductive plates applied to the human body according to a selected operation mode to provide the effect of medium or low frequency wave massage, which can be utilized in rehabilitation and fat-reduction exercises.

The secondary objective of the present invention is to provide a binding cloth for medium or low frequency wave massage, wherein a plurality of conductive buttons can be deployed along each conductive strip; the conductive buttons are each covered by an insulating cloth cover. A cloth cover can be lifted open so that a conductive plate can be buckled up thereon. The conductive plates that are not connected to conductive buttons are not electrically charged and do not provide the electro-stimulating massage. This allows the selection of a variety of body regions to be massaged.

The various objects and advantages of the present invention will be more readily understood from the following detailed description when read in conjunction with the appended drawings.

### BRIEF DESCRIPTION OF THE DRAWINGS

FIG. 1 is a perspective view of the present invention embodied in a belt.

FIG. 2 is an exploded perspective view of the conductive strips and the associated conductive buttons of the present invention.

FIG. 3 is a front perspective view of the controller of the present invention.

FIG. 4 is a back perspective view of the controller of the present invention.

FIG. 5 is a cross-sectional view of the controller being buckled up with the binding cloth.

FIG. 6 shows the connectivity of the central integrated circuit of the controller of the present invention.

FIG. 7 is the circuit layout of the controller of the present invention.

FIG. 8 is a diagram showing the present invention embodied in an elbow guard.

FIG. 9 is a diagram showing the present invention embodied in a knee guard.

FIG. 10 is a diagram showing the present invention embodied in binding cloths for other body parts.

FIG. 11 is a diagram showing the present invention embodied in a waist binding cloth.

FIG. 12 shows the conductive buttons on a binding cloth covered by an overlapped two-piece cloth cover.

### DETAILED DESCRIPTION OF THE PREFERRED EMBODIMENTS

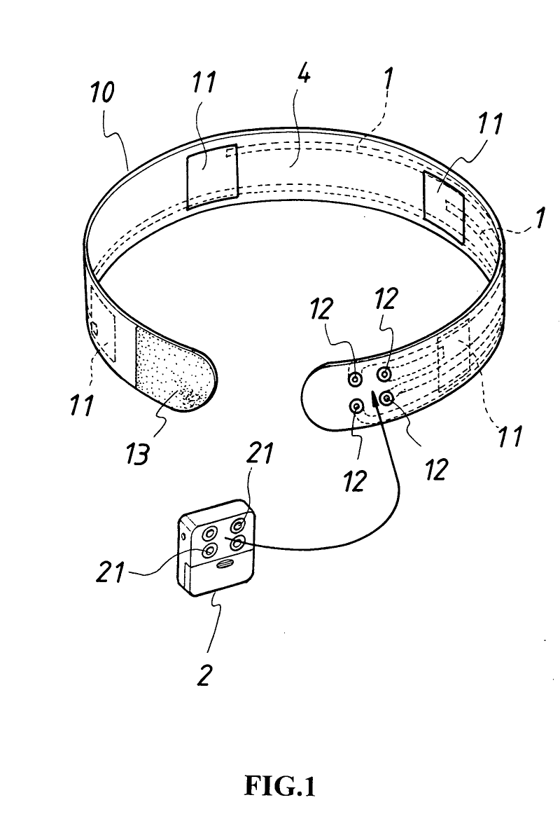

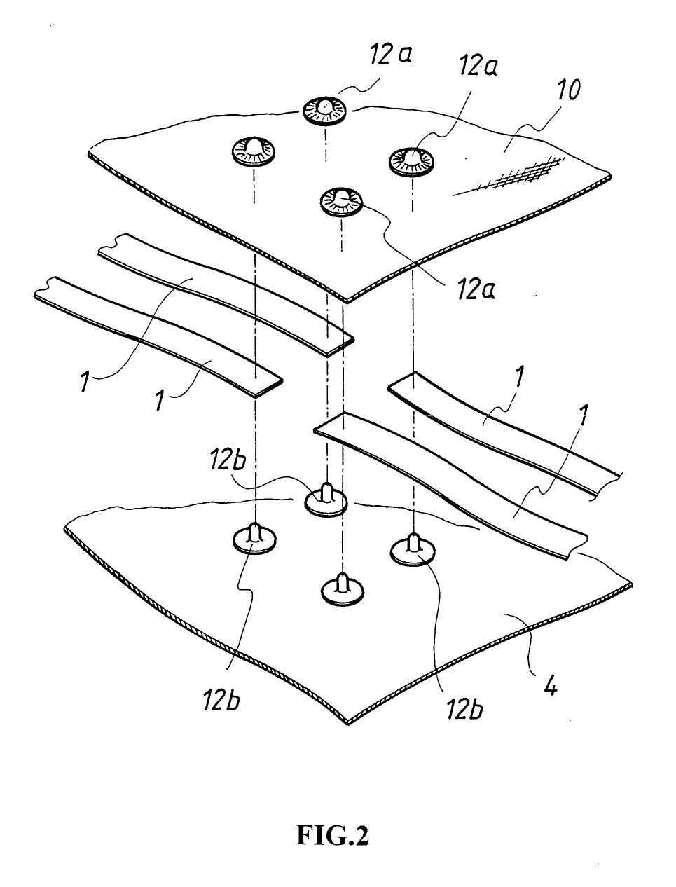

Referring to FIG. 1 and FIG. 2, the present invention as a binding cloth for medium or low frequency wave massage comprises a plurality of conductive strips 1 enclosed in a binding cloth 10 on selected locations, each having a first end connected to a conductive plate 11 and a second end connected to a conductive button 12. The conductive buttons 12 are concentrated on a selected location of the binding cloth 10 and then may engaged with a plurality of corresponding conductive buttons 21 on an electro-stimulating controller 2 so that the controller is combined with the binding cloth 10. When charged, the conductive plates 11 in contact with the human body provide the effect of medium or low frequency wave massage.

The above-mentioned conductive strips 1 and conductive plates 11 are seamed into the binding cloth 10 in the way that the conductive strips 1 are hidden within the binding cloth 10, whereas the conductive plates 11 are uncovered so as to provide direct contact with the human body. The binding cloth 10 can be an annular object, which is formed by linking two ends of the binding cloth 10 with an adhesive unit 13.

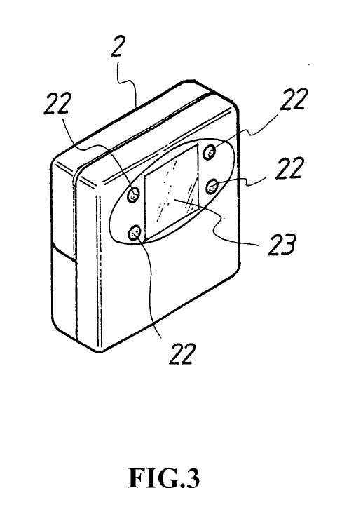

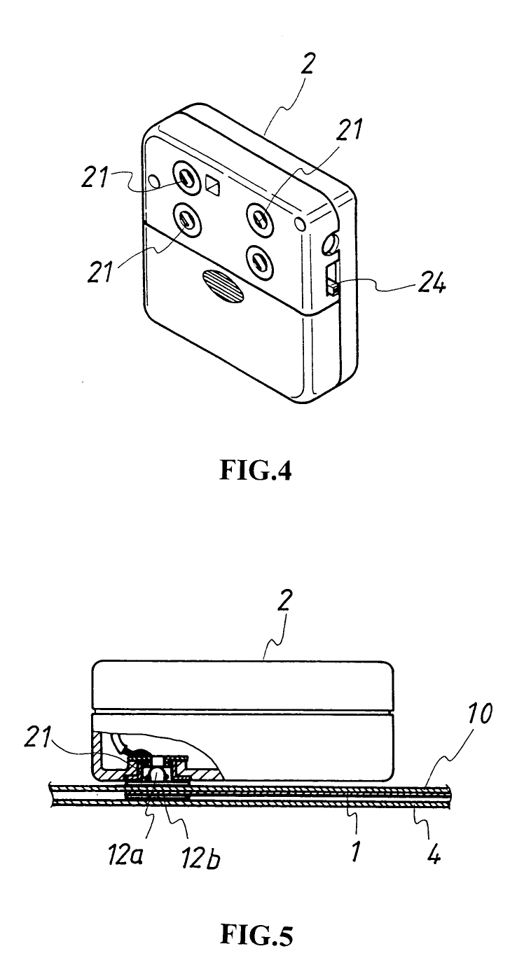

Referring to FIG. 2, the conductive buttons 12 to which the second ends of the conductive strips 1 are connected each comprises a female piece 12a and a male piece 12b. Each pair of a female piece 12a and a male piece 12b is rivet-jointed so that the corresponding conductive strip 1 is sandwiched by them and thus secured within the binding cloth 10. The conductive buttons 12, concentrated on a selected location of the binding cloth 10 and uncovered by the cloth, can be buckled up with a plurality of corresponding conductive buttons 21 (as shown in FIG. 3) so as to mount the electro-stimulating controller 2 to the binding cloth 10 (as shown in FIG. 4).

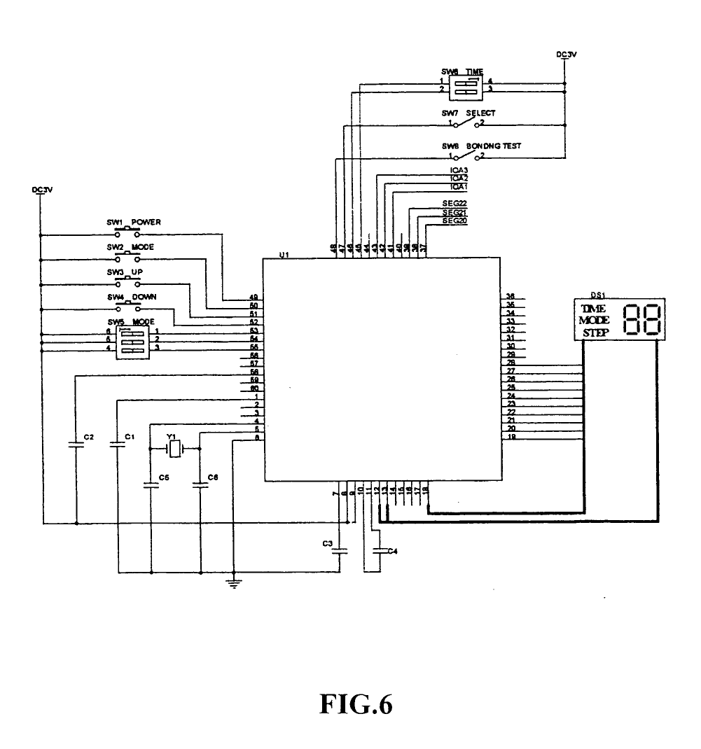

The electro-stimulating controller 2 is provided with a central IC (integrated circuit) for transporting a pulsed wave that periodically charges and discharges the associated inductors and capacitors. To produce massage effects of various degrees, the bandwidth of the pulsed wave can be changed by a program over a range from 1 Hz to 150 Hz. The electro-stimulating controller 2 includes a plurality of control buttons 22 for respectively adjusting the amplitude of electric currents, operating time, power on/off and massage modes. The electro-stimulating controller 2 further includes an LCD panel 23 for displaying the operation status. The pulsed high-voltage signal, produce by the internal circuit as shown in FIG. 5 and FIG. 6, are directed to a plurality of conductive buttons 21 to form an output terminal. One lateral side of the electro-stimulating controller 2 is provided with a stepping switch 24 for selecting members of the output terminal to be charged.

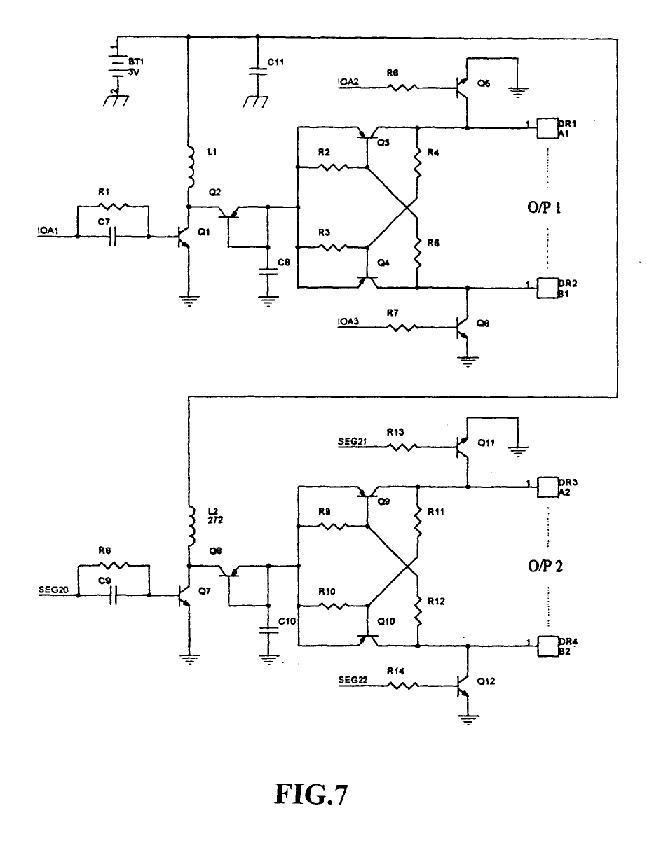

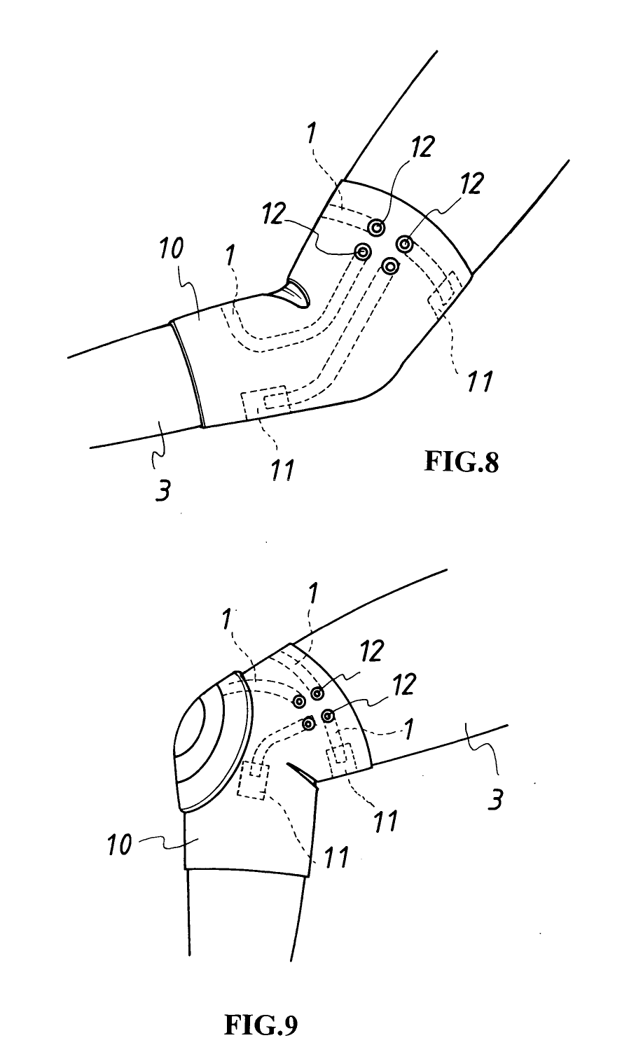

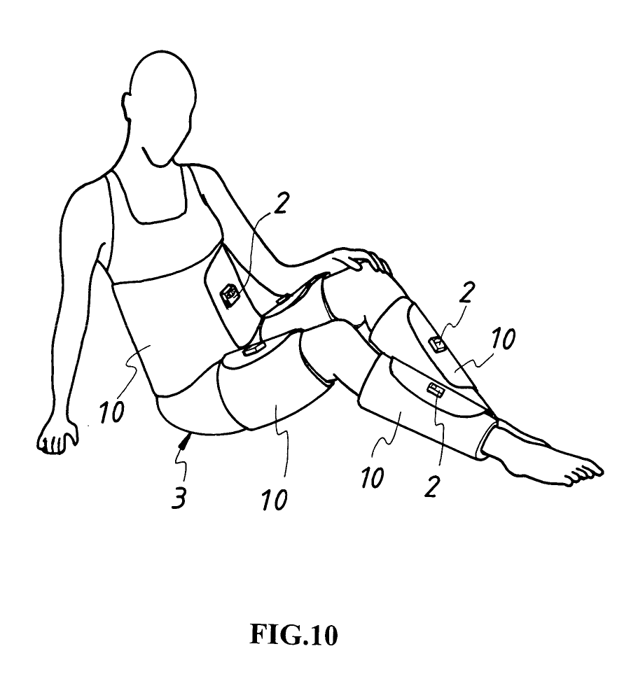

Unrestricted by shapes and sizes, the binding cloth of the present invention can be embodied in a belt as shown in FIG. 1, an elbow guard as shown in FIG. 7, a knee guard as shown in FIG. 8 and a waist band as shown in FIG. 9 and FIG. 10. The binding cloths 10 in the above the preferred embodiments all have a plurality of conductive strips 1, with both ends of each of them respectively connected to a conductive plate 11 and a conductive button 12. The conductive plates 11 provide direct contact with a human body 3. The conductive buttons 12 are concentrated on a selected location of the binding cloth 10, which can be coupled with a plurality of conductive buttons 21 on an electro-stimulating controller 2. The binding cloth 10 can be applied to various parts of the human body 3, according to the operation modes selected by the electro-stimulating controller 2. A binding cloth for medium or low frequency wave massage thus designed has an effect of medium or low frequency wave massage that is healthful to the human body. It can also be utilized in rehabilitation exercises and fat reduction.

Referring to FIG. 10, another preferred embodiment of the present invention has a binding cloth 10 wherein each conductive strip 1 is provided with one or more than one conductive buttons 14. The conductive buttons 14 are distributed according to the acupuncture points or fat-accumulative places in the human body. Each of the conductive buttons 14 is capable of being rivet-connected with another conductive button 111, which is coupled to a conductive plate 11. Each of the conductive buttons 14 is covered with a cloth cover 4 that can be opened when a conductive button 111 is applied to. To activate the electro-stimulating effect of a conductive plate 11, a conductive button 111 is firstly mounted onto a conductive button 14, which is then connected to the conductive plate 11 so that the associated conductive strip 1 becomes closed. The conductive plates 11 that are not connected to conductive buttons 14 are therefore not electrically charged and do not provide the electro-stimulating massage. This allows the selection of a variety of body regions to be massaged.

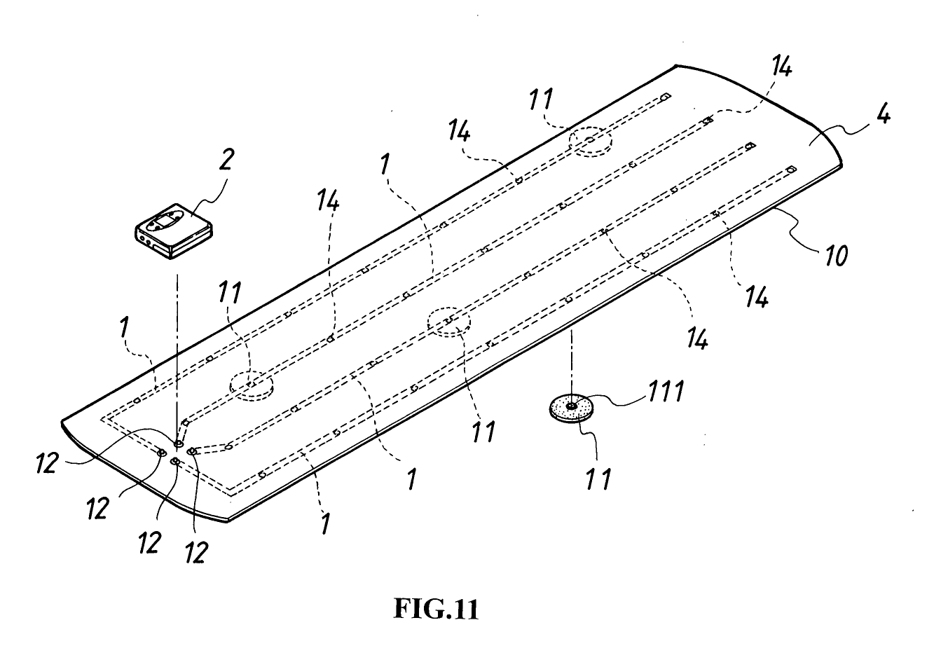

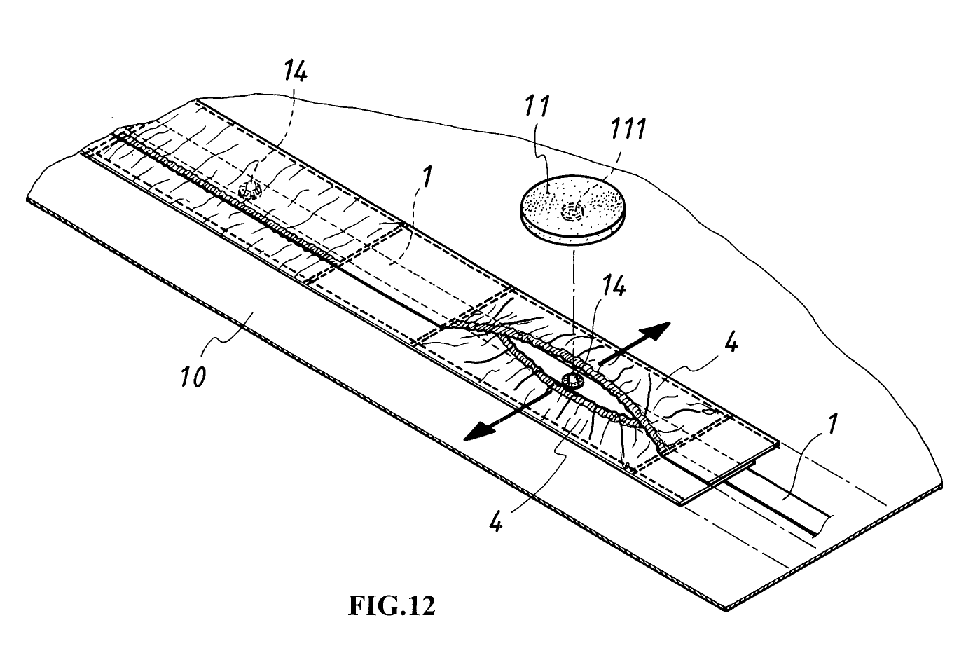

Referring to FIG. 11, the above-mentioned conductive buttons 14 on the binding cloth 10 are each covered by a cloth cover 4 formed by a left piece and a right piece that are overlapped to hide the associated conductive button 14, which is thereby electrically insolated. To use a conductive button 14, the left and right pieces of a cloth cover 4 are pushed aside to enable a connection with a conductive plate 11 of a conductive strip 1. The cloth cover 4 can also be of one piece, which is lifted when the conductive button 14 underneath is to be used.

The conductive strip 1 of the present invention is made of ordinary electro-conductive materials, and preferably a soft electro-conductive cloth.

The present invention is thus described, and it will be obvious that the same may be varied in many ways. Such variations are not to be regarded as a departure from the spirit and scope of the present invention, and all such modifications as would be obvious to one skilled in the art are intended to be included within the scope of the following claims.

1. A binding cloth for medium or low frequency wave massage comprising:
a binding cloth;
a plurality of conductive strips each having a first end connected with a conductive plate and a second end with a conductive button, said conductive buttons being concentrated on a selected location on said binding cloth; and
an electro-stimulating controller having a plurality of conductive buttons capable of being buckled up with said conductive buttons on said binding cloth, said controller thereby being capable of being mounted on said binding cloth;
said electro-stimulating controller charging said conductive plates through said conductive strips so as to provide an effect of electro-stimulating massage to body parts said conductive plates in contact with.

2. The binding cloth for medium or low frequency wave massage of claim 1, wherein said conductive strips and said conductive plates are seamed into said binding cloth so that said conductive strips are secured within said binding cloth; a surface of each said conductive plate is not covered by said binding cloth for being nakedly applied on the human body.

3. The binding cloth for medium or low frequency wave massage of claim 1, wherein said binding cloth is an annular object.

4. The binding cloth for medium or low frequency wave massage of claim 1, wherein two ends of said binding cloth is capable of being jointed by an adhesive unit to form an annular object.

5. The binding cloth for medium or low frequency wave massage of claim 1, wherein said controller is provided with a central integrated circuit for transporting a pulsed wave train that periodically charges and discharges a plurality of inductors and capacitors; to produce massage effects of various degrees, the bandwidth of said pulsed wave train can be changed by a built-in program.

6. The binding cloth for medium or low frequency wave massage of claim 5, wherein the bandwidth of said pulsed wave train ranges from 1 Hz to 150 Hz.

7. The binding cloth for medium or low frequency wave massage of claim 1, wherein said controller is for providing a plurality of conductive buttons with high voltage to form an output terminal.

8. The binding cloth for medium or low frequency wave massage of claim 1, wherein said controller is provided with a stepping switch on a lateral side thereof for selecting a group of said conductive buttons to be charged.

9. The binding cloth for medium or low frequency wave massage of claim 1, wherein said binding cloth is embodied in a form selected from a belt, an elbow guard, a knee guard and a waist binding cloth.

10. The binding cloth for medium or low frequency wave massage of claim 1, wherein at least one conductive button is deployed close to the conductive-plate end of each said conductive strip;
said conductive buttons are deployed according to acupuncture points or fat-accumulative places of the human body; each of said conductive buttons is capable of being rivet-connected with a conductive button that is coupled to one of said conductive plates.

11. The binding cloth for medium or low frequency wave massage of claim 10, wherein each of said conductive buttons deployed on said conductive strips is covered with an insulating cloth cover.

12. The binding cloth for medium or low frequency wave massage of claim 11, wherein each of said cloth covers takes a form selected from a single piece and an overlapped two-piece set.

13. The binding cloth for medium or low frequency wave massage of claim 1, wherein said conductive strips are made of conductive cloth.
